(19) United States
(12) Patent Application Publication
(10) Pub. No.: US 2025/0327017 A1
(43) Pub. Date: Oct. 23, 2025

(54) Systems and Methods of Liquid Submerged Fermentation Using Liquid Dilution Spawning for Expanding Inoculum of Fungal Species

(71) Applicant: M2 Ingredients, Inc., Vista, CA (US)

(72) Inventors: Julie Daoust, Vista, CA (US); Samuel Andrasko, Lakeside, CA (US)

(21) Appl. No.: 19/096,553

(22) Filed: Mar. 31, 2025

Related U.S. Application Data
(60) Provisional application No. 63/636,605, filed on Apr. 19, 2024.

(51) Int. Cl. C12N 1/14; C12M 1/00; C12M 1/12; C12M 1/34; C12R 1/645

(52) U.S. Cl. CPC C12N 1/14; C12M 23/26; C12M 29/00; C12M 37/06; C12M 41/42; C12R 2001/645

(57) ABSTRACT

Embodiments relate to systems and methods for liquid submerged fermentation to expand inoculum of fungal species, addressing the technical problem of lengthy production times in fungal culture. The solution involves a method comprising inoculating growth media in a sterilized container, agitating the media under optimized conditions, diluting the fungal species in a secondary container, and inoculating bioreactor bags to increase biomass volume. This process enables even colonization and consolidation of the fungal species, resulting in full spectrum fungal biomass production. Embodiments include efficient production of fungal biomass with reduced lead time and labor costs compared to standard systems. Embodiments are applicable to various fungal genera, utilizing specific growth media compositions and controlled environmental conditions to optimize growth and production.

### RELATED APPLICATION

This application claims the priority benefit of U.S. Provisional Patent Application Ser. No. 63/636,605 filed on Apr. 19, 2024 and titled “Systems and Methods of Liquid Submerged Fermentation for Expanding Inoculum of Fungal Species.” The aforementioned disclosure is hereby incorporated by reference in its entirety for all purposes.

### FIELD OF THE TECHNOLOGY

Embodiments of the disclosure relate to liquid submerged fermentation. In particular, the present disclosure relates to systems and methods of liquid submerged fermentation using liquid dilution spawning for expanding inoculum of fungal species to produce full spectrum fungal biomass in a production environment.

### BACKGROUND

The approaches described in this section could be pursued, but are not necessarily approaches that have previously been conceived or pursued. Therefore, unless otherwise indicated, it should not be assumed that any of the approaches described in this section qualify as prior art merely by virtue of their inclusion in this background section.

The production of fungal biomass plays a significant role in various industries, including pharmaceuticals, agriculture, and food production. Traditional cultivation methods often involve large-scale equipment and direct media inoculation, which require considerable financial investment and extensive infrastructure. These conventional approaches are not only costly but also inefficient, as they typically involve lengthy production times and labor-intensive processes. Furthermore, the requirement for substantial volumes of an initial expanding culture can be inefficient, limiting scalability and flexibility in production.

Current methods face challenges in efficiently expanding small volumes of high-density fungal cultures into larger quantities of biomass. This inefficiency leads to increased production costs and extended lead times, posing significant barriers to meeting the growing demand for fungal biomass. The absence of streamlined processes for rapid and cost-effective expansion of fungal cultures underscores the need for innovative solutions that can reduce initial investment, optimize resource use including multiple quality control checks, and enhance the overall efficiency of high quality fungal biomass production.

### SUMMARY

The present technology as described herein uses liquid submerged fermentation for expanding inoculum of fungal species to produce full spectrum fungal biomass in a production environment.

In some embodiments, the present technology relates to a method of liquid submerged fermentation using liquid dilution spawning for expanding inoculum of fungal species to produce full spectrum fungal biomass in a production environment, the method including: starting a mother culture of a target fungal species; inoculating growth media in a first sterilized container with the target fungal species of the mother culture for liquid submerged fermentation; agitating the growth media in the first sterilized container using optimized growth conditions for the target fungal species of the mother culture; diluting the target fungal species in a second sterilized container by diluting the growth media from the first sterilized container including the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

In some embodiments, the present technology relates to a method, further including placing the plurality of bioreactor bags in climate-controlled rooms to complete a growth cycle of the target fungal species to produce full spectrum fungal biomass.

In some embodiments, the present technology relates to a method further comprising a first quality control check, the first quality control check being during the diluting the target fungal species in the second sterilized for each lot and being checking the first sterilized container for contamination and quality of the target fungal species to avoid contamination for each lot.

In some embodiments, the present technology relates to a method, further including a second quality control check, the second quality control check being checking the plurality of bioreactor bags for contamination and quality of the target fungal species and verifying successful colonization of the target fungal species in each bioreactor bag of the plurality of bioreactor bags to avoid contamination before proceeding to a growth phase.

In some embodiments, the present technology relates to a method, further including agitating each bioreactor bag of the plurality of bioreactor bags post-inoculation to ensure even distribution of diluted liquid inoculum throughout the growth media.

In some embodiments, the present technology relates to a method, wherein the even colonization of the target fungal species in the second sterilized container is uniform distribution and growth of the target fungal species throughout the second sterilized container.

In some embodiments, the present technology relates to a method, wherein the enabling consolidation of the target fungal species in the second sterilized container includes the target fungal species growing and integrating into a cohesive mass within the second sterilized container.

In some embodiments, the present technology relates to a method, wherein the even dispersion of fragments of mycelium of the liquid inoculum on the plurality of grain media dispersion points inside of the plurality of bioreactor bags includes uniformly distributing the fragments of mycelium across a plurality of designated points within each bioreactor bag, the plurality of grain media dispersion points being inoculation sites of the target fungal species that facilitate rapid and consistent colonization of the target fungal species.

In some embodiments, the present technology relates to a method, wherein the consolidation of the target fungal species in the plurality of bioreactor bags is the target fungal species integrating into a cohesive and uniform mass in each of the plurality of bioreactor bags.

In some embodiments, the present technology relates to a method, wherein the inoculating the plurality of bioreactor bags for increasing the biomass volume of full spectrum fungal biomass of the target fungal species using the quantity of liquid inoculum from the second sterilized container enables even distribution of the target fungal species throughout each bioreactor bag of the plurality of bioreactor bags facilitating rapid and consistent colonization by the target fungal species within each bioreactor bag of the plurality of bioreactor bags.

In some embodiments, the present technology relates to a method, wherein the first sterilized container is a primary carboy, the primary carboy including a stir bar and a draw tube for aseptic transfer of diluted liquid inoculum to the second sterilized container.

In some embodiments, the present technology relates to a method, wherein the second sterilized container is a secondary carboy, the secondary carboy including a stir bar and a draw tube for aseptic transfer of diluted liquid inoculum to the plurality of bioreactor bags.

In some embodiments, the present technology relates to a method, wherein the draw tube includes a dispensing tube located on a dispensing end of the draw tube coupled with a bioreactor bag of the plurality of bioreactor bags; wherein the second sterilized container is coupled with a bump switch.

In some embodiments, the present technology relates to a method, wherein the plurality of bioreactor bags are inoculated using a peristaltic pump for even distribution of diluted liquid inoculum.

In some embodiments, the present technology relates to a method, wherein the growth media in the first sterilized container includes a mixture of cauliflower powder, dextrose, yeast extract, and other nutrients tailored to specific needs of the target fungal species.

In some embodiments, the present technology relates to a method, wherein the enabling consolidation of the target fungal species in the plurality of bioreactor bags includes the target fungal species growing and integrating into a cohesive mass within the plurality of bioreactor bags.

In some embodiments, the present technology relates to a method, wherein the first sterilized container is a primary carboy, the primary carboy including a shaft including impellers for agitating the growth media at an optimal speed ranging from 50 to 350 RPM, the optimal speed depending on the target fungal species.

In some embodiments, the present technology relates to a method, wherein the primary carboy includes a variable frequency drive (VFD) controlled motor to adjust the optimal speed of the impellers, the optimal speed of the impellers optimizing agitation conditions for the target fungal species.

In some embodiments, the present technology relates to a method of liquid submerged fermentation using liquid dilution spawning for expanding inoculum of fungal species to produce full spectrum fungal biomass in a production environment, the method including: inoculating growth media in a first sterilized container with a target fungal species of a mother culture for liquid submerged fermentation; agitating the growth media in the first sterilized container using optimized growth conditions for the target fungal species of the mother culture; diluting the target fungal species in a second sterilized container by diluting the growth media from the first sterilized container including the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

In some embodiments, the present technology relates to a method of liquid submerged fermentation using liquid dilution spawning for expanding inoculum of fungal species to produce full spectrum fungal biomass in a production environment, the method including: inoculating growth media in a first sterilized container with a target fungal species of a mother culture for liquid submerged fermentation; agitating the growth media in the first sterilized container using optimized growth conditions for the target fungal species of the mother culture; diluting the target fungal species in a second sterilized container by diluting the growth media from the first sterilized container including the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and inoculating a bioreactor bag for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the bioreactor bag, the plurality of grain media dispersion points being a plurality of inoculation points in the bioreactor bag for increasing fungal biomass volume enabling even colonization of the target fungal species in the bioreactor bag thereby enabling consolidation of the target fungal species in the bioreactor bag, the increasing fungal biomass volume resulting in full spectrum fungal biomass production including target compounds.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 shows a first diagram of an exemplary system including a primary carboy and a secondary carboy allowing for a secondary dilution for expanding inoculum of fungal species using liquid submerged fermentation and liquid dilution spawning to produce full spectrum fungal biomass in a production environment, according to various embodiments of the present technology.

FIG. 2 shows a second diagram of an exemplary system including a secondary carboy, a laminar flow hood, and a bioreactor bag for expanding inoculum of fungal species using liquid submerged fermentation and liquid dilution spawning of an expanded inoculum to produce full spectrum fungal biomass in a production environment, according to various embodiments of the present technology.

FIG. 3 illustrates a comparative block diagram of an exemplary system including a primary carboy, a secondary carboy for a secondary dilution, and bioreactor bags for expanding inoculum of fungal species using liquid submerged fermentation and liquid dilution spawning to produce full spectrum fungal biomass in a production environment compared to a standard large scale bioreactor system, according to various embodiments of the present technology.

FIG. 4 shows another comparative block diagram of a bioreactor bag inoculated from the secondary carboy by using liquid submerged fermentation and liquid dilution spawning for enabling even colonization of the target fungal species in the plurality of bioreactor bags thereby enabling consolidation of the target fungal species in the plurality of bioreactor bags compared to a standard system, according to various embodiments of the present technology.

FIG. 5 shows comparative graphs depicting a decrease in the initial investment and cost of goods per year of liquid submerged fermentation using liquid dilution spawning for expanding inoculum of fungal species to produce full spectrum fungal biomass in a production environment compared to a standard system, according to various embodiments of the present technology.

FIG. 6 shows a comparative graph depicting a decrease in lead time of liquid submerged fermentation using liquid dilution spawning for expanding inoculum of fungal species to produce full spectrum fungal biomass in a production environment compared to a standard system for grain spawn preparation, according to various embodiments of the present technology.

FIG. 7 shows another comparative graph depicting a decrease in labor of liquid submerged fermentation using liquid dilution spawning for expanding inoculum of fungal species to produce full spectrum fungal biomass in a production environment compared to a standard system for grain spawn preparation, according to various embodiments of the present technology.

FIG. 8 show an exemplary method of liquid submerged fermentation using liquid dilution spawning for expanding inoculum of fungal species to produce full spectrum fungal biomass in a production environment, according to various embodiments of the present technology.

### DETAILED DESCRIPTION

In the following description, for purposes of explanation, numerous specific details are set forth in order to provide a thorough understanding of the disclosure. It will be apparent, however, to one skilled in the art, that the disclosure may be practiced without these specific details. In other instances, structures and devices may be shown in block diagram form only in order to avoid obscuring the disclosure. It should be understood that the disclosed embodiments are merely exemplary of the invention, which may be embodied in multiple forms. Those details disclosed herein are not to be interpreted in any form as limiting, but as the basis for the claims.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Various exemplary embodiments described and illustrated herein relate to systems and methods of submerged fermentation for expanded inoculum of fungal species to produce full spectrum fungal biomass in a production environment.

The production of fungal biomass plays a significant role in various industries, such as pharmaceuticals, agriculture, and food production. Traditional methods of fungal cultivation often involve complex and labor-intensive procedures, which can lead to inefficiencies and increased costs. These conventional approaches typically require large-scale systems that are not only costly to set up but also pose challenges in maintaining suitable conditions for diverse fungal species. Additionally, the transfer of fungal cultures from one medium to another can be cumbersome, resulting in extended production times and potential contamination risks.

Current systems often struggle with achieving uniform colonization and consolidation of fungal species, which are necessary for producing high-quality biomass. The lack of efficient methods to expand cultures and optimize conditions further exacerbates these challenges, leading to inconsistent production outcomes. As industries continue to demand more efficient and reliable methods for fungal biomass production, there is a pressing need for innovative solutions that streamline the cultivation process, reduce lead times, and minimize labor costs while ensuring the production of high-quality fungal biomass.

The present technology addresses these challenges by introducing systems and methods for liquid submerged fermentation, specifically designed to expand inoculum of fungal species and produce full spectrum fungal biomass in a production environment. The present technology leverages liquid dilution spawning techniques to enhance the efficiency of fungal biomass production, offering a streamlined process that reduces lead times and labor costs. By optimizing growth conditions and ensuring even colonization and consolidation of fungal species, the disclosed systems and methods of the present technology provide a reliable and cost-effective solution for producing high-quality fungal biomass across various industries. Unlike traditional methods that require large reactors and significant investment, some embodiments of the present technology utilize a dilution step that allows for the use of smaller initial volumes of culture, which are then expanded into larger volumes. This process involves the use of a higher concentration of liquid inoculum, enabling even dispersion of mycelium fragments across multiple grain media dispersion points. This results in a higher number of initial inoculant points within the bioreactor bags, facilitating quicker colonization and reducing the time required for full consolidation of the fungal biomass. The present technology not only reduces the cost and resources needed for large-scale production but also enhances the efficiency of the inoculation process, making it a significant improvement over existing techniques.

A problem in production fungal culture is a large time period for production including using fermentation of a fungal culture in a large container and then transferring the fungal culture to bioreactor bags. In some embodiments, the present technology solves this problem by using dilution of the fungal culture in a secondary container to decrease the time period for production resulting in a volume of mycelium using expanded inoculum of a target fungal species to produce full spectrum fungal biomass in a production environment.

Exemplary embodiments of the present technology include the following steps according to various embodiments. In some embodiments, a stock mother culture is retrieved from cold storage. For example, an exemplary method includes starting a mother culture of a target fungal species.

The present technology includes but is not limited to a stock mother culture of species within the following Genera: Ganoderma, Morchella, Grifola, Pleurotus, Lentinula, Cordyceps, Ophiocordyceps, Calocybe, Hypsizygus, Tolypocladium, Phellinus, Inonotus, Trametes, Laricifomes, Fomes, Agaricus, Antrodia, Laetiporus, Polyporus, Lepista, Auricularia, Stropharia, Hericium, Pholiota, Wolfiporia, Agrocybe, Sporassis, Phallus, Xylaria, Flammulina, Volvariella, Macrolepiota, Lepiota, Coprinus, Tramella, Globiformes, Lignosus, Phallus, or Psilocybe (hereinafter “Genera of Stock Mother Cultures”). For example, a stock mother culture of the Genera of Stock Mother Cultures may be transferred to a one-hundred mm petri dish comprising media, the media comprising but not limited to water, cauliflower powder, dextrose monohydrate, nutritional yeast, potato starch, potato peptone #2, carrot powder, carob powder, l-glutamine, L-lysine, Hypoxanthine, calcium carbonate, calcium sulfate, magnesium sulfate, activated carbon, whole grain oat powder, and rice protein supplement.

In various embodiments, the cultures are monitored for inconsistencies in growth. For instance, irregularities in growth, DNA damage, and senescence may reduce the performance of the culture. This growth period may be from five days to three weeks (21 days) depending on the fungal species (e.g., each Genus of the Genera of Stock Mother Cultures may have different growth times). The healthiest culture representing the correct characteristics of the original mother culture may be selected to be used for an inoculum.

In some embodiments, five 50 ml centrifuge tubes of liquid media comprising, but not limited to, water, cauliflower powder, dextrose monohydrate, yeast extract, potato starch, peptone, carrot powder, carob powder, l-glutamine, L-lysine, Hypoxanthine, calcium carbonate, calcium sulfate, magnesium sulfate, activated carbon, powder of whole grains, or rice protein supplement may be sterilized in an autoclave for forty-five minutes at fifteen PSI to render all organisms inactive and then allowed to cool to eighty degrees Fahrenheit.

In some embodiments, five 50 ml centrifuge tubes of liquid media, the liquid media comprising, but not limited to, water, cauliflower powder, dextrose monohydrate, yeast extract, potato starch, peptone, carrot powder, carob powder, l-glutamine, L-lysine, Hypoxanthine, calcium carbonate, calcium sulfate, magnesium sulfate, activated carbon, powder of whole grains, or rice protein supplement is sterilized in an autoclave for forty-five minutes at 15 PSI to render all organism inactive and allowed to cool to eighty degrees Fahrenheit.

In some embodiments, the selected culture is inoculated into the 50 ml centrifuge tubes aseptically in front of a HEPA filtrated laminar flow hood.

In various embodiments after inoculation of the selected culture into the 50 ml centrifuge tubes, the 50 ml centrifuge tubes may be placed onto a vertically oriented rotator and rotated for one to three weeks as required, dependent on the fungal species (e.g., each Genus of the Genera of Stock Mother Cultures may have a different growth time).

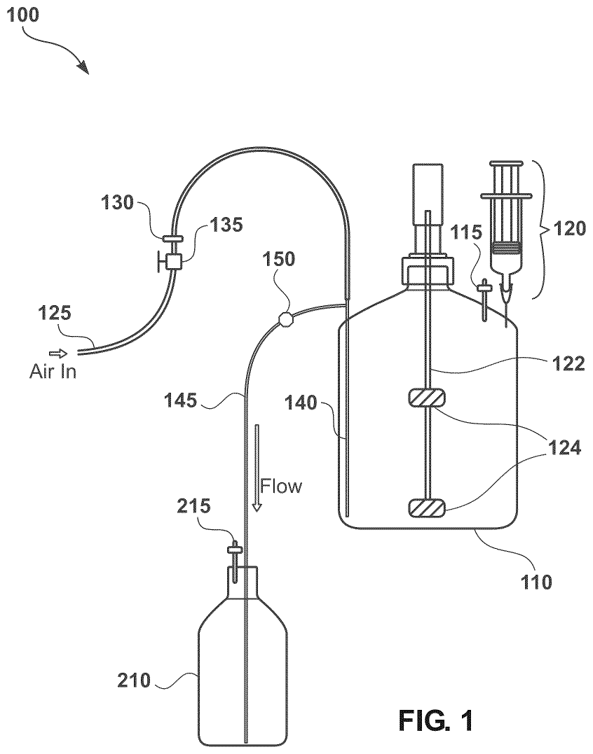

According to various embodiments, once the selected culture has been grown out thoroughly, the inoculum is ready to transfer into a twenty Liter to one-hundred Liter primary carboy (e.g., a primary carboy 110 shown in FIG. 1). Sizing of the primary carboy depends on the amount of production bags to be produced. If the selected cultures are not used immediately, the selected cultures may be placed into cold storage until needed. For example, one to five 50 ml centrifuge tubes may be used for the inoculum. For example, the number of 50 ml centrifuge tubes is dependent on the density of the growth in each culture.

FIG. 1 shows a first diagram of an exemplary system 100 including a primary carboy 110 and a secondary carboy 210 allowing for a secondary dilution for expanding inoculum of fungal species using liquid submerged fermentation and liquid dilution spawning to produce full spectrum fungal biomass in a production environment, according to various embodiments of the present technology. FIG. 1 shows the exemplary system 100 including the primary carboy 110 for expanding inoculum of fungal species using liquid submerged fermentation to produce full spectrum fungal biomass in a production environment, according to various embodiments of the present technology. FIG. 1 shows the primary carboy 110 including a 0.2 micron filter 115, a syringe with inoculum 120 of a fungal species from a mother culture, a shaft 122 with attached impellers 124 for agitation. FIG. 1 further shows the primary carboy 110 coupled with an air tube 125 (for air supply), a 0.2 micron filter 130, a stopcock 135, and a down tube 140 into the primary carboy 110. FIG. 1 also shows the primary carboy 110 coupled with a draw tube 145 including a connection 150. The draw tube 145 couples the primary carboy 110 with the secondary carboy 210 that includes a 0.2 micron filter 215.

In some embodiments, the primary carboy 110 is prepared for expanding the selected fungal species. For example, the primary carboy 110 may be from twenty Liters to one-hundred Liters in volume. In some embodiments, the primary carboy 110 used with exemplary system 100 shown in FIG. 1 may be fitted with a 0.2 micron inline filter (e.g., the 0.2 micron filter 115) and down tube 140 for gas exchange by a pump with a manual or automatic proportioning valve to control the rate of gas exchange or lock the air feed (air supply from the air tube 125) for using the draw tube 145. The primary carboy 110 is also fitted with an autoclavable head assembly which is attached to the lid of the primary carboy 110, complete with seals, and a shaft with attached impellers 124 for agitation of the media. A Variable Frequency Drive (VFD) controlled motor is attached after inoculation to supply the optimal speed (e.g., 50 to 350 Revolutions Per Minute (RPM)) of agitation for the fungal species being cultured within the vessel without the introduction of contaminates. The speed of agitation is dependent on the species being grown. (e.g., each Genus of the Genera of Stock Mother Cultures may have different speed of agitation). For example, the growth behavior of some fungal species are different under different agitation conditions. For example, some fungal species produce undesirable growth characteristics with non-optimal agitation conditions. Consequently, the agitation conditions including the rate at which each fungal species is agitated may be critical, in various embodiments. For example, pelletizing of mycelium is an undesirable characteristic that may be caused by non-optimal agitation conditions. For example, during optimal agitation condition the mycelium is loose and dispersed in smaller fragments, which is important for fungal species growth.

In some embodiments, the primary carboy 110 is assembled complete with the autoclavable head assembly. The primary carboy 110 may include the down tube 140 and the air tube 125 (for air supply), which may be a silicone supply tube for the inclusion of air through the down tube 140. The primary carboy 110 further includes a 0.2 micron filter 115, aeration block at the end of the down tube 140. The draw tube 145 may be silicone tube that enables the sanitary removal of the fungal species being cultured from the primary carboy 110 once the culture fermentation is complete. The attached tubing (e.g., down tube 140 and draw tube 145) may be connected via TC ports, barbed fittings and luer lock disconnects.

In some embodiments, the primary carboy 110 is assembled and filled with water and a growth medium. The growth medium may include media comprising but not limited to, cauliflower powder, dextrose, sucrose, lactose, maltose, yeast extract, potato starch, Tapioca starch, peptone, carrot powder, carob powder, l-glutamine, L-lysine, Hypoxanthine, calcium carbonate, calcium sulfate, magnesium sulfate, activated carbon, and powder of whole grains, (or rice protein supplement). The growth medium may be optimized for each species. In other words, each fungal species may have different growth medium in some embodiments.

In some embodiments, the primary carboy 110 is loaded into an autoclave complete with the autoclavable head assembly and liquid media. The primary carboy 110 may be autoclaved for 1.5 to 3.5 hours at two-hundred-and-fifty degrees Fahrenheit (fifteen PSI) depending on the volume of the carboy.

In various embodiments, the primary carboy 110 is cooled inside a HEPA filtrated clean room to below eighty degrees Fahrenheit.

In some embodiments, the exemplary method includes inoculating a first sterilized container comprising growth media with the target fungal species for submerged fermentation, the target fungal species being of the mother culture. For example, once cooled, the syringe with inoculum 120 (inoculum of the target fungal species) is loaded with the culture and injected into the primary carboy 110 by means of a luer lock fitting mounted on the primary carboy 110 in front of a laminar flow hood 205. For example, inoculating growth media in a first sterilized container with the target fungal species of the mother culture for liquid submerged fermentation.

In some embodiments, the exemplary method includes agitating the growth media comprising the target fungal species in the first sterilized container using optimized growth conditions for the target fungal species of the mother culture. For example, an electric motor is attached to the head assembly on the primary carboy 110 which activates the shaft 122 with the attached impellers 124 for agitation of media including the inoculum of the target fungal species using the optimal speed (e.g., 50-350 RPM) of agitation for the target fungal species being cultured. (e.g., each Genus of the Genera of Stock Mother Cultures may have different optimal agitation conditions). For example, agitating the growth media in the first sterilized container using optimized growth conditions for the target fungal species of the mother culture.

In some embodiments, the fermentation of the species of fungus proceeds for the duration needed for each fungal species (e.g., each Genus of the Genera of Stock Mother Cultures may have different fermentation times). Air exchange is provided though the 0.2 micron filter 215 and through using the air tube 125 as well as the down tube 140 that may be equipped with a stainless aeration stone.

In some embodiments, upon the completion of the fermentation, the secondary carboy 210 (e.g., a twenty Liter carboy) is equipped with a stir bar, a draw tube 145 (e.g., silicone tubing), a 0.2 micron filter 215 for inclusion of filtered air, and sanitary connections to accept fluid aseptically to avoid contamination. The secondary carboy 210 is filled with ten Liters to sixteen Liters of water and is autoclaved for 3.5 hours at two-hundred-and-fifty degrees Fahrenheit (15 PSI). The volume of water is dependent on the fungal species used and the recovery rate needed (e.g., each Genus of the Genera of Stock Mother Cultures may have a different optimal volume of water).

In some embodiments, the secondary carboy 210 is cooled inside a HEPA filtrated room until the temperature is below eighty Fahrenheit before the secondary carboy 210 is attached to the draw tube 145 (e.g., feed tube).

In some embodiments, the exemplary method includes diluting the target fungal species using a second sterilized container (e.g., the secondary carboy 210) by diluting a volume of the growth media from the first sterilized container (e.g., the primary carboy 110) comprising the fungal species of the mother culture, the diluting the target fungal species enabling use of a small amount of the volume of the growth media from the first sterilized container (e.g., the primary carboy 110) for dilution. For instance, diluting the target fungal species in a second sterilized container (e.g., the secondary carboy 210) by diluting the growth media from the first sterilized container (e.g., the primary carboy 110) comprising the target fungal species of the mother culture, the diluting the target fungal species using a volume of the growth media from the first sterilized container (e.g., the primary carboy 110) resulting in a concentration of the target fungal species in the second sterilized container (e.g., the secondary carboy 210) that enables even colonization of the target fungal species in the second sterilized container thereby enabling consolidation of the target fungal species in the second sterilized container (e.g., the secondary carboy 210). For example, once cooled, the secondary carboy 210 is attached to the draw tube 145 (e.g., feed tube) of the primary carboy 110 and the secondary carboy 210 accepts four to ten Liters of the fermented mycelium creating a dilution ratio of 4:1 to 2:1. This ratio is dependent on the fungal species and the recovery rate needed (e.g., each Genus of the Genera of Stock Mother Cultures may have a different optimal dilution ratio).

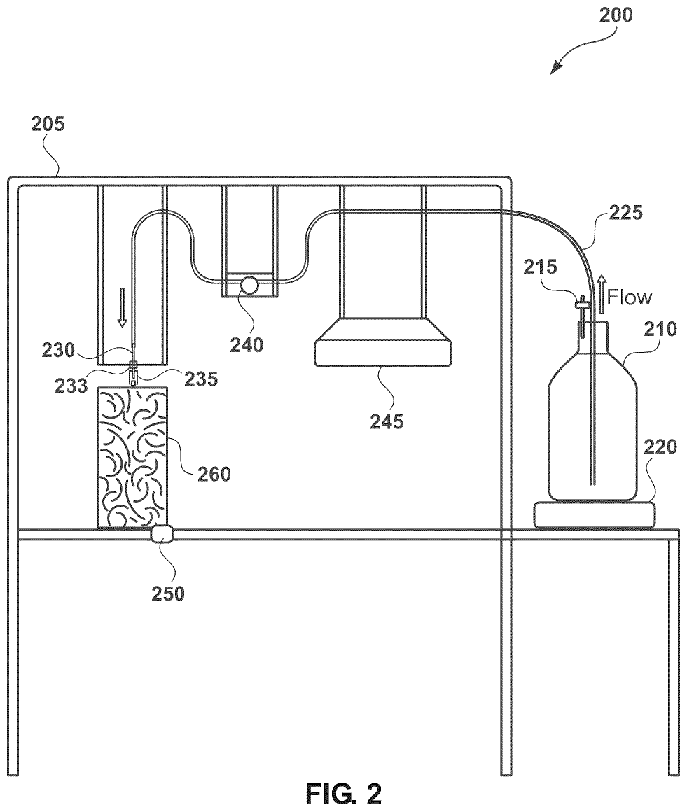

Turning to FIG. 2, FIG. 2 shows a second diagram of an exemplary system 200 including the secondary carboy 210, a laminar flow hood 205, and a bioreactor bag 260 for expanding inoculum of fungal species using liquid submerged fermentation and liquid dilution spawning of an expanded inoculum to produce full spectrum fungal biomass in a production environment, according to various embodiments of the present technology. FIG. 2 shows the second diagram of the exemplary system 200 including the secondary carboy 210, the laminar flow hood 205, and the bioreactor bag 260 for expanding inoculum of fungal species using a dilution of an expanded inoculum to produce full spectrum fungal biomass in a production environment, according to various embodiments of the present technology.

According to various embodiments, the secondary carboy 210 includes the 0.2 micron filter 215 and is placed on a stir plate 220. A flow hood draw tube 225 couples the bioreactor bag 260 to the secondary carboy 210. The exemplary system 200 of FIG. 2 further shows a laminar flow hood 205 covering a sealer 245, a peristaltic pump 240 coupled with the flow hood draw tube 225, and a stainless steel dispensing tube 230 on the dispensing end of the flow hood draw tube 225 including a male luer lock fitting 235 and two stainless steel pipe collets 233. The stainless steel dispensing tube 230 is coupled with the bioreactor bag 260 and a bump switch 250.

In some embodiments, the secondary carboy 210 is fitted with the flow hood draw tube 225 (e.g., a ten foot sterilized silicone draw tube). On the end of the flow hood draw tube 225 is a stainless steel dispensing tube 230 (e.g., stainless steel six inch long tube that is fitted to the inside of the flow hood draw tube 225) (e.g., silicone tubing) for dispensing the diluted inoculum. For example, a two-inch piece of the silicone tube may be fitted onto the end of the stainless steel dispensing tube 230 and a male luer lock fitting 235 may be fitted to the two-inch piece of silicone tubing on the end of the stainless steel dispensing tube 230 for dispensing the inoculum into the final growth media in the bioreactor bag 260. For example, the male luer lock fitting 235 narrows the opening for which the inoculum gets dispensed so as to prevent the inoculum from dripping from the stainless steel dispensing tube 230. In some embodiments, a tapered stainless steel tube may also be used in place of the male luer lock fitting 235.

In various embodiments, the flow hood draw tube 225 (e.g., a ten foot sterilized silicone draw tube) is hung along the upper struts of a laminar flow hood 205 out of the way and the dispensing end of the flow hood draw tube 225 (e.g., stainless steel dispensing tube 230) is mounted to a plate over the inoculation area at a height determined by the height of the bioreactor bag 260 that is to be inoculated (as shown in FIG. 2). This is done by installing two stainless steel pipe collets 233 spaced to the width of the bracing plate so that the flow hood draw tube 225 can slide onto a channel cut into the bracing plate to be securely attached.

In various embodiments, a peristaltic pump 240, is mounted onto a shelf inside the laminar flow hood 205, that accepts the flow hood draw tube 225 and is set to a pre-determined inoculation rate. This pre-determined inoculation rate may be from 700:1 to 100:1 by volume in various embodiments. The bump switch 250 mounted on the table or floor in front of the laminar flow hood 205 allows a user (e.g., an inoculation technician) to trigger each predetermined amount of inoculum into each bioreactor bag 260 successively. For example, the secondary carboy 210 may be placed onto a stir plate 220 outside the laminar flow hood 205 and the media in the secondary carboy 210 is rotated at approximately two-hundred RPM to keep the fungal species being cultivated in suspension.

In some embodiments, the exemplary method includes inoculating a plurality of bioreactor bags for increasing a biomass volume of full spectrum fungal biomass of the target fungal species using a quantity of liquid inoculum from the second sterilized container (e.g., the secondary carboy 210), the quantity of liquid inoculum from the second sterilized container (e.g., the secondary carboy 210) enabling an even dispersion of fragments of mycelium of the liquid inoculum on a plurality of grain media dispersion points inside of the plurality of bioreactor bags (e.g., the bioreactor bag 260), the plurality of grain media dispersion points being a plurality of inoculation points in each bioreactor bag (e.g., the bioreactor bag 260) for increasing fungal biomass volume enabling even colonization of the target fungal species in the plurality of bioreactor bags thereby enabling consolidation of the target fungal species in the plurality of bioreactor bags (e.g., the bioreactor bag 260), the increasing fungal biomass volume resulting in full spectrum fungal biomass production including target compounds. For example, a user (e.g., an inoculation technician), one-by-one, moves each sterilized bioreactor bag (e.g., the bioreactor bag 260 that is sterilized) full of growth media under the stainless steel dispensing tube 230 and triggers the peristaltic pump 240 with the bump switch 250. Each bioreactor bag 260 is then sealed using the sealer 245, and the media is then shaken to thoroughly spread the inoculum through the media. Next, each bioreactor bag 260 may be placed into climate controlled rooms for the remainder of the growth cycle and fruited to produce full spectrum fungal biomass in the production environment.

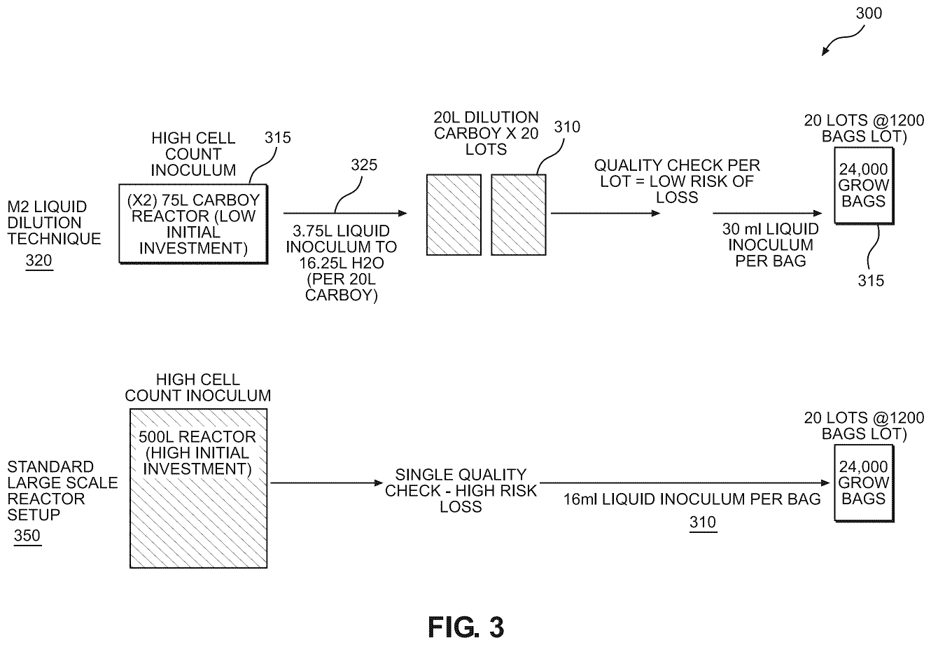

FIG. 3 illustrates a comparative block diagram 300 of an exemplary system including a primary carboy 305 (e.g., the primary carboy 110), a secondary carboy 310 (e.g., the secondary carboy 210) for a secondary dilution, and bioreactor bags 315 for expanding inoculum of fungal species using liquid submerged fermentation and liquid dilution spawning 320 to produce full spectrum fungal biomass in a production environment compared to a standard large scale bioreactor system 350, according to various embodiments of the present technology. The expanding inoculum of fungal species using liquid submerged fermentation and liquid dilution spawning 320 of the present technology includes the secondary dilution 325 that enables even colonization of the target fungal species in the second sterilized container (e.g., the secondary carboy 310) thereby allowing consolidation of the target fungal species in the second sterilized container (e.g., the secondary carboy 310). In contrast, the standard large scale bioreactor system 350 does not include the secondary dilution 325.

According to various embodiments, expanding inoculum of fungal species using liquid submerged fermentation and liquid dilution spawning 320 may begin with two 75 L primary carboys (the primary carboy 305 which is a low initial investment compared to the standard large scale bioreactor system 350 that includes a five-hundred Liter reactor) that are inoculated with the target fungal species of the mother culture for liquid submerged fermentation including a high cell count inoculum. The present technology includes a secondary dilution 325. For example, the expanding inoculum of fungal species using liquid submerged fermentation and liquid dilution spawning 320 may use 3.75 L of liquid inoculum into 16.25 L of media for a twenty Liter carboy (e.g., the secondary carboy 310) for the secondary dilution 325. For instance, twenty lots of twenty Liter carboys has a low risk of loss because a quality check is possible for each lot. For example, the expanding inoculum of fungal species using liquid submerged fermentation and liquid dilution spawning 320 may use 30 mL of liquid inoculum per bioreactor bag from the secondary dilution 325 into twenty lots of 1200 bioreactor bags per lot for 24,000 bioreactor grow bags (the bioreactor bags 315).

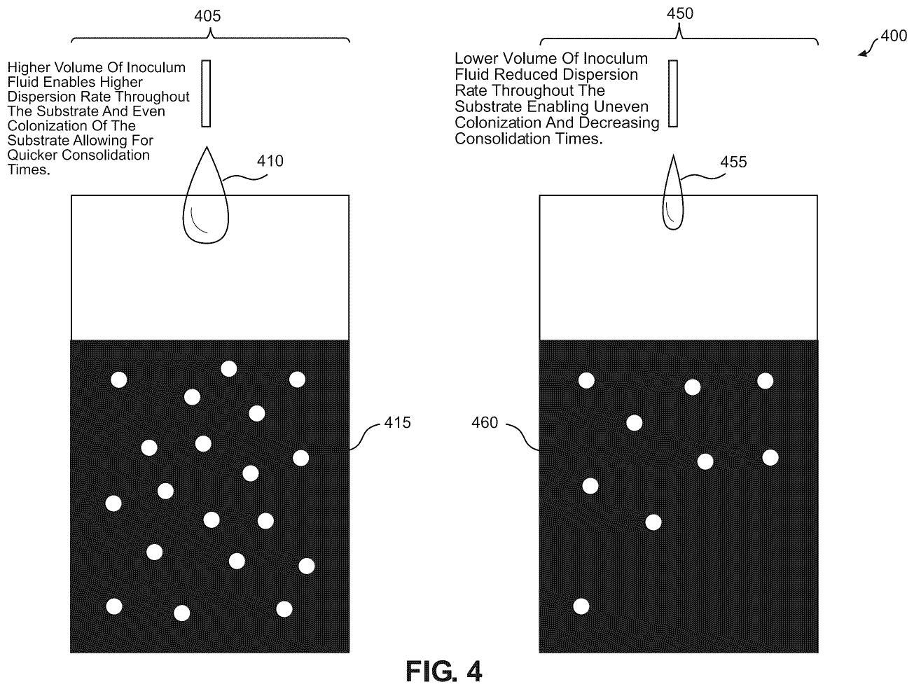

In contrast, the standard large scale bioreactor system 350 includes a five-hundred Liter container (which is a high initial investment compared to the present technology that may use two 75 L primary carboys (the primary carboy 305)) and the standard large scale bioreactor system 350 does not include the secondary dilution 325. For example, the standard large scale bioreactor system 350 uses sixteen mL of liquid inoculum for dilution per bioreactor bag. For example, sixteen mL of liquid inoculum per bioreactor bag into twenty lots of 1200 bioreactor bags per lot for 24,000 bioreactor grow bags. A downside of the standard large scale bioreactor system 350 is that there is a single quality check and high risk of loss. In contrast, according to some embodiments the present technology includes liquid submerged fermentation using liquid dilution spawning 320 allows for a quality check per lot enabling a low risk of loss. Furthermore, using liquid submerged fermentation and liquid dilution spawning 320 enables even colonization of the target fungal species in the secondary carboy 310 (e.g., the second sterilized container) thereby allowing consolidation of the target fungal species in the secondary carboy 310 (e.g., the second sterilized container) to produce full spectrum fungal biomass in a production environment compared to the standard large scale bioreactor system 350 as described further in FIG. 4.

FIG. 4 shows another comparative block diagram 400 of a bioreactor bag 405 inoculated from the secondary carboy (e.g., the secondary carboy 210) by using liquid submerged fermentation and liquid dilution spawning 320 (shown in FIG. 3) for enabling even colonization of the target fungal species in the plurality of bioreactor bags thereby enabling consolidation of the target fungal species in the plurality of bioreactor bags compared to a standard system 450, according to various embodiments of the present technology. According to some embodiments, FIG. 4 illustrates expanding inoculum of fungal species using liquid submerged fermentation and liquid dilution spawning 320 (shown in FIG. 3) in the bioreactor bag 405 compared with the standard system 450 (e.g., the standard large scale bioreactor system 350). FIG. 4 shows, for example, a higher volume of inoculum fluid 410 that enables higher dispersion rate 415 the substrate and even colonization of the substrate allowing for quicker consolidation times, compared with the standard system 450. In contrast, a lower volume of inoculum fluid 455 results in a lower dispersion rate 460 (i.e., reduces dispersion rate) throughout the substrate enabling uneven colonization and decreasing consolidation times using the standard system 450, compared to using liquid submerged fermentation and liquid dilution spawning.

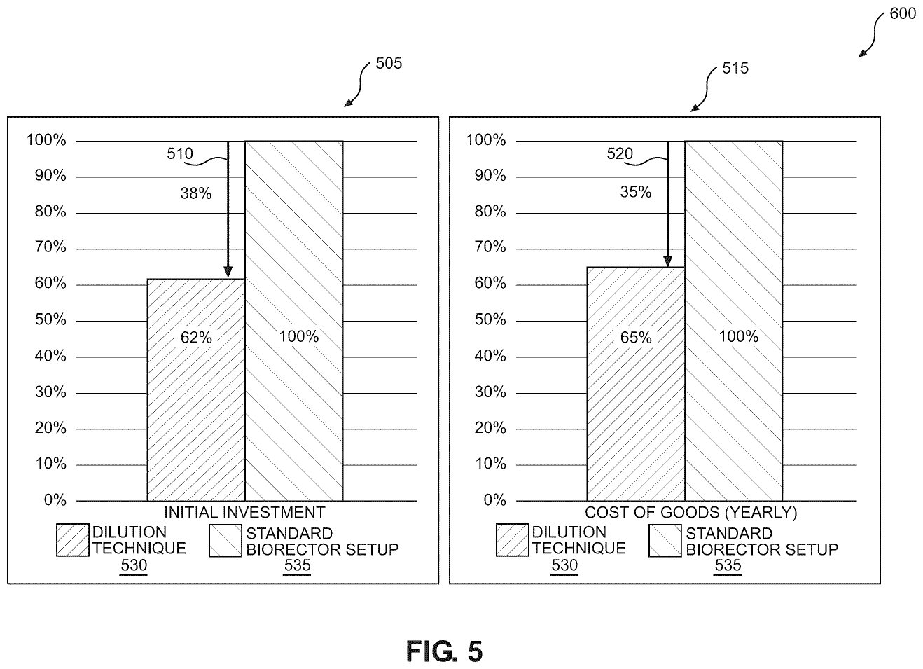

FIG. 5 shows comparative graphs 500 depicting a decrease in the initial investment and cost of goods per year by using liquid submerged fermentation and liquid dilution spawning for expanding inoculum of fungal species to produce full spectrum fungal biomass in a production environment compared to a standard system, according to various embodiments of the present technology. For example, the comparative graphs 500 of FIG. 5 illustrates a initial investment graph 505 and a cost of goods per year graph 515. The initial investment graph 505 shows a thirty-eight percent decrease 510 in the cost as calculated by savings in lab materials used compared with a standard bioreactor system. For example, the comparative graphs 500 of FIG. 5 further illustrate the cost of goods per year graph 515. The cost of goods per year graph 515 shows a thirty-five percent decrease 520 in the cost of good as calculated by savings in lab materials used by liquid submerged fermentation using liquid dilution spawning 530 compared with a standard bioreactor system 535.

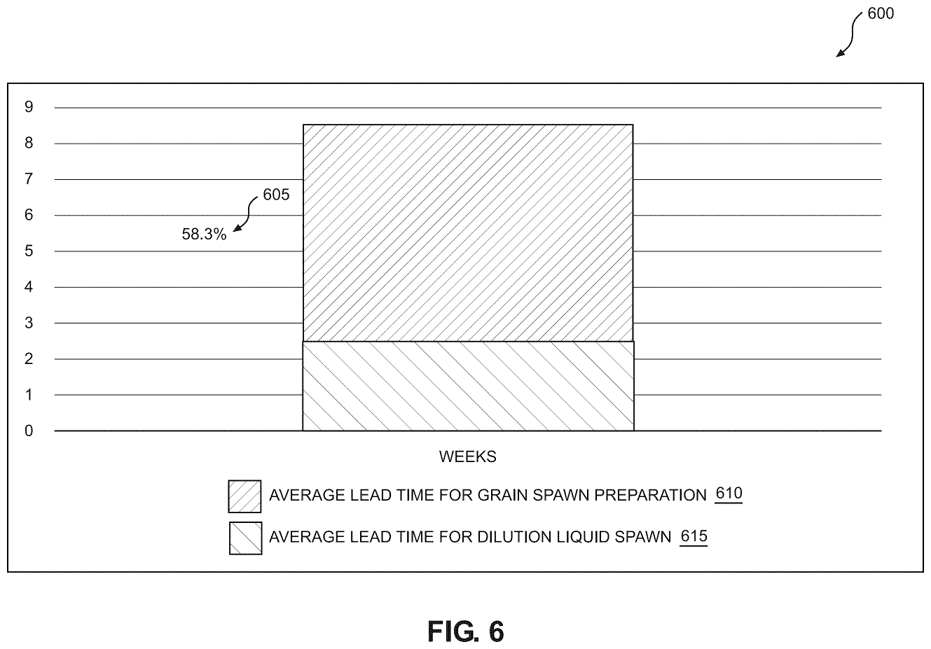

FIG. 6 shows a comparative graph 600 depicting a decrease in lead time of liquid submerged fermentation using liquid dilution spawning for expanding inoculum of fungal species to produce full spectrum fungal biomass in a production environment compared to a standard system for grain spawn preparation, according to various embodiments of the present technology. For example, the comparative graph 600 further shows a 58.3 percent saving 605 in lead time compared with standard grain spawn preparation 610 by using liquid submerged fermentation using liquid dilution spawning 615.

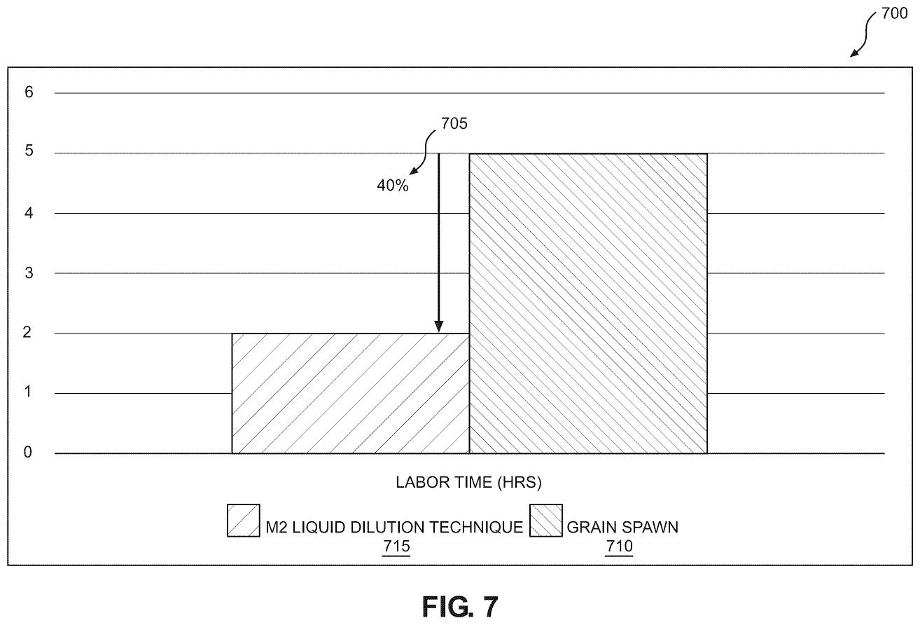

FIG. 7 shows another comparative graph 700 depicting a decrease in labor of liquid submerged fermentation using liquid dilution spawning for expanding inoculum of fungal species to produce full spectrum fungal biomass in a production environment compared to a standard system for grain spawn preparation, according to various embodiments of the present technology. For example, the comparative graph 700 shows a forty percent saving 705 in labor compared with standard grain spawn preparation 710 by using liquid submerged fermentation using liquid dilution spawning 715.

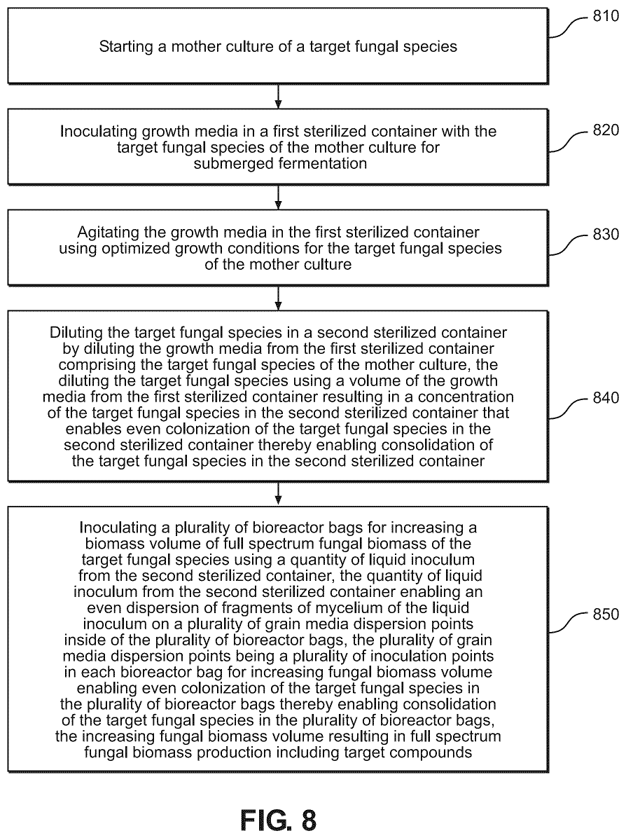

FIG. 8 show an exemplary method of liquid submerged fermentation using liquid dilution spawning for expanding inoculum of fungal species to produce full spectrum fungal biomass in a production environment, according to various embodiments of the present technology. According to some embodiments, FIG. 8 is a flowchart of an example method for liquid submerged fermentation using liquid dilution spawning for expanding inoculum of fungal species to produce full spectrum fungal biomass in a production environment, the method comprising the following steps.

According to some embodiments, liquid dilution spawning is a method used in fungal cultivation where a small volume of high-density fungal culture is diluted with a liquid medium to expand the inoculum into larger quantities. This technique facilitates the even distribution of fungal mycelium across a substrate, enhancing colonization and consolidation, and ultimately increasing the production efficiency of fungal biomass.

According to some embodiments, at step 810, starting a mother culture of a target fungal species. At step 820, inoculating growth media in a first sterilized container with the target fungal species of the mother culture for liquid submerged fermentation. At step 830, agitating the growth media in the first sterilized container using optimized growth conditions for the target fungal species of the mother culture,

According to some embodiments, at step 840, diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container.

According to some embodiments, the process of diluting the target fungal species in a second sterilized container involves transferring a portion of the growth media containing the fungal species from a first sterilized container to a second one. This dilution reduces the concentration of the fungal species, allowing for more even distribution and colonization within the second container. The goal is to achieve a uniform spread of the fungal species, which facilitates their growth and integration into a cohesive mass, thereby enhancing the efficiency and effectiveness of the fungal biomass production process. Even colonization of the target fungal species in the second sterilized container refers to the uniform distribution and growth of the fungal species throughout the container. This uniform colonization ensures that the fungal species can effectively integrate and form a cohesive mass, known as consolidation, within the container. The process facilitates optimal growth conditions, leading to efficient production of fungal biomass.

According to some embodiments, at step 850, inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

According to some embodiments, inoculating a plurality of bioreactor bags involves introducing a liquid inoculum containing fragments of mycelium into multiple bioreactor bags to increase the biomass volume of a target fungal species. This process uses a liquid inoculum from a second sterilized container to ensure even dispersion of the mycelium fragments across various grain media dispersion points within each bag. The goal is to achieve uniform colonization and consolidation of the fungal species, resulting in the production of full spectrum fungal biomass, which includes the desired target compounds.

According to some embodiments, enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds refers to a process in which a fungal species is uniformly distributed and grows consistently across multiple bioreactor bags. This uniform colonization ensures that the fungal species can effectively integrate and form a cohesive mass within each bag, leading to an increase in fungal biomass. The result is the production of a comprehensive range of fungal biomass that includes the desired target compounds, optimizing the yield and quality of the biomass produced.

According to some embodiments, a method of liquid submerged fermentation using liquid dilution spawning for expanding inoculum of fungal species to produce full spectrum fungal biomass in a production environment, the method comprising: starting a mother culture of a target fungal species; inoculating growth media in a first sterilized container with the target fungal species of the mother culture for liquid submerged fermentation; agitating the growth media in the first sterilized container using optimized growth conditions for the target fungal species of the mother culture; diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

According to some embodiments, further comprising placing the plurality of bioreactor bags in climate-controlled rooms to complete a growth cycle of the target fungal species to produce full spectrum fungal biomass.

According to some embodiments, further comprising a first quality control check, the first quality control check being during the diluting the target fungal species in the second sterilized for each lot and being checking the first sterilized container for contamination and quality of the target fungal species to avoid contamination for each lot.

According to some embodiments, further comprising a second quality control check, the second quality control check being checking the plurality of bioreactor bags for contamination and quality of the target fungal species and verifying successful colonization of the target fungal species in each bioreactor bag of the plurality of bioreactor bags to avoid contamination before proceeding to a growth phase.

According to some embodiments, further comprising agitating each bioreactor bag of the plurality of bioreactor bags post-inoculation to ensure even distribution of diluted liquid inoculum throughout the growth media.

According to some embodiments, wherein the even colonization of the target fungal species in the second sterilized container is uniform distribution and growth of the target fungal species throughout the second sterilized container.

According to some embodiments, wherein the enabling consolidation of the target fungal species in the second sterilized container comprises the target fungal species growing and integrating into a cohesive mass within the second sterilized container.

According to some embodiments, wherein the even dispersion of fragments of mycelium of the liquid inoculum on the plurality of grain media dispersion points inside of the plurality of bioreactor bags comprises uniformly distributing the fragments of mycelium across a plurality of designated points within each bioreactor bag, the plurality of grain media dispersion points being inoculation sites of the target fungal species that facilitate rapid and consistent colonization of the target fungal species.

According to some embodiments, wherein the consolidation of the target fungal species in the plurality of bioreactor bags is the target fungal species integrating into a cohesive and uniform mass in each of the plurality of bioreactor bags.

According to some embodiments, wherein the inoculating the plurality of bioreactor bags for increasing the biomass volume of full spectrum fungal biomass of the target fungal species using the quantity of liquid inoculum from the second sterilized container enables even distribution of the target fungal species throughout each bioreactor bag of the plurality of bioreactor bags facilitating rapid and consistent colonization by the target fungal species within each bioreactor bag of the plurality of bioreactor bags.

According to some embodiments, wherein the first sterilized container is a primary carboy, the primary carboy comprising a stir bar and a draw tube for aseptic transfer of diluted liquid inoculum to the second sterilized container.

According to some embodiments, wherein the second sterilized container is a secondary carboy, the secondary carboy comprising a stir bar and a draw tube for aseptic transfer of diluted liquid inoculum to the plurality of bioreactor bags.

According to some embodiments, wherein the draw tube comprises a dispensing tube located on a dispensing end of the draw tube coupled with a bioreactor bag of the plurality of bioreactor bags; wherein the second sterilized container is coupled with a bump switch.

According to some embodiments, wherein the plurality of bioreactor bags are inoculated using a peristaltic pump for even distribution of diluted liquid inoculum.

According to some embodiments, wherein the growth media in the first sterilized container comprises a mixture of cauliflower powder, dextrose, yeast extract, and other nutrients tailored to specific needs of the target fungal species.

According to some embodiments, wherein the enabling consolidation of the target fungal species in the plurality of bioreactor bags comprises the target fungal species growing and integrating into a cohesive mass within the plurality of bioreactor bags.

According to some embodiments, wherein the first sterilized container is a primary carboy, the primary carboy comprising a shaft comprising impellers for agitating the growth media at an optimal speed ranging from 50 to 350 RPM, the optimal speed depending on the target fungal species.

According to some embodiments, wherein the primary carboy comprises a variable frequency drive (VFD) controlled motor to adjust the optimal speed of the impellers, the optimal speed of the impellers optimizing agitation conditions for the target fungal species.

According to some embodiments, a method of liquid submerged fermentation using liquid dilution spawning for expanding inoculum of fungal species to produce full spectrum fungal biomass in a production environment, the method comprising: inoculating growth media in a first sterilized container with a target fungal species of a mother culture for liquid submerged fermentation; agitating the growth media in the first sterilized container using optimized growth conditions for the target fungal species of the mother culture; diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

According to some embodiments, a method of liquid submerged fermentation using liquid dilution spawning for expanding inoculum of fungal species to produce full spectrum fungal biomass in a production environment, the method comprising: inoculating growth media in a first sterilized container with a target fungal species of a mother culture for liquid submerged fermentation; agitating the growth media in the first sterilized container using optimized growth conditions for the target fungal species of the mother culture; diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and inoculating a bioreactor bag for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the bioreactor bag, the plurality of grain media dispersion points being a plurality of inoculation points in the bioreactor bag for increasing fungal biomass volume enabling even colonization of the target fungal species in the bioreactor bag thereby enabling consolidation of the target fungal species in the bioreactor bag, the increasing fungal biomass volume resulting in full spectrum fungal biomass production including target compounds.

Thus, the present technology including systems and methods of liquid submerged fermentation for expanding inoculum of fungal species to produce full spectrum fungal biomass in a production environment is disclosed. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method of liquid submerged fermentation using liquid dilution spawning for expanding inoculum of fungal species to produce full spectrum fungal biomass in a production environment, the method comprising:
starting a mother culture of a target fungal species;
inoculating growth media in a first sterilized container with the target fungal species of the mother culture for liquid submerged fermentation;
agitating the growth media in the first sterilized container using optimized growth conditions for the target fungal species of the mother culture;
diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and
inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

2. The method of claim 1, further comprising placing the plurality of bioreactor bags in climate-controlled rooms to complete a growth cycle of the target fungal species to produce full spectrum fungal biomass.

3. The method of claim 1, further comprising a first quality control check, the first quality control check being during the diluting the target fungal species in the second sterilized for each lot and being checking the first sterilized container for contamination and quality of the target fungal species to avoid contamination for each lot.

4. The method of claim 1, further comprising a second quality control check, the second quality control check being checking the plurality of bioreactor bags for contamination and quality of the target fungal species and verifying successful colonization of the target fungal species in each bioreactor bag of the plurality of bioreactor bags to avoid contamination before proceeding to a growth phase.

5. The method of claim 1, further comprising agitating each bioreactor bag of the plurality of bioreactor bags post-inoculation to ensure even distribution of diluted liquid inoculum throughout the growth media.

6. The method of claim 1, wherein the even colonization of the target fungal species in the second sterilized container is uniform distribution and growth of the target fungal species throughout the second sterilized container.

7. The method of claim 1, wherein the enabling consolidation of the target fungal species in the second sterilized container comprises the target fungal species growing and integrating into a cohesive mass within the second sterilized container.

8. The method of claim 1, wherein the even dispersion of fragments of mycelium of the liquid inoculum on the plurality of grain media dispersion points inside of the plurality of bioreactor bags comprises uniformly distributing the fragments of mycelium across a plurality of designated points within each bioreactor bag, the plurality of grain media dispersion points being inoculation sites of the target fungal species that facilitate rapid and consistent colonization of the target fungal species.

9. The method of claim 1, wherein the consolidation of the target fungal species in the plurality of bioreactor bags is the target fungal species integrating into a cohesive and uniform mass in each of the plurality of bioreactor bags.

10. The method of claim 1, wherein the inoculating the plurality of bioreactor bags for increasing the biomass volume of full spectrum fungal biomass of the target fungal species using the quantity of liquid inoculum from the second sterilized container enables even distribution of the target fungal species throughout each bioreactor bag of the plurality of bioreactor bags facilitating rapid and consistent colonization by the target fungal species within each bioreactor bag of the plurality of bioreactor bags.

11. The method of claim 1, wherein the first sterilized container is a primary carboy, the primary carboy comprising a stir bar and a draw tube for aseptic transfer of diluted liquid inoculum to the second sterilized container.

12. The method of claim 1, wherein the second sterilized container is a secondary carboy, the secondary carboy comprising a stir bar and a draw tube for aseptic transfer of diluted liquid inoculum to the plurality of bioreactor bags.

13. The method of claim 12, wherein the draw tube comprises a dispensing tube located on a dispensing end of the draw tube coupled with a bioreactor bag of the plurality of bioreactor bags; wherein the second sterilized container is coupled with a bump switch.

14. The method of claim 1, wherein the plurality of bioreactor bags are inoculated using a peristaltic pump for even distribution of diluted liquid inoculum.

15. The method of claim 1, wherein the growth media in the first sterilized container comprises a mixture of cauliflower powder, dextrose, yeast extract, and other nutrients tailored to specific needs of the target fungal species.

16. The method of claim 1, wherein the enabling consolidation of the target fungal species in the plurality of bioreactor bags comprises the target fungal species growing and integrating into a cohesive mass within the plurality of bioreactor bags.

17. The method of claim 1, wherein the first sterilized container is a primary carboy, the primary carboy comprising a shaft comprising impellers for agitating the growth media at an optimal speed ranging from 50 to 350 RPM, the optimal speed depending on the target fungal species.

18. The method of claim 17, wherein the primary carboy comprises a variable frequency drive (VFD) controlled motor to adjust the optimal speed of the impellers, the optimal speed of the impellers optimizing agitation conditions for the target fungal species.

19. A method of liquid submerged fermentation using liquid dilution spawning for expanding inoculum of fungal species to produce full spectrum fungal biomass in a production environment, the method comprising:
inoculating growth media in a first sterilized container with a target fungal species of a mother culture for liquid submerged fermentation;
agitating the growth media in the first sterilized container using optimized growth conditions for the target fungal species of the mother culture;
diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and
inoculating a plurality of bioreactor bags for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the plurality of bioreactor bags, the plurality of grain media dispersion points being a plurality of inoculation points in each bioreactor bag for increasing fungal biomass volume enabling even colonization of the target fungal species in the plurality of bioreactor bags thereby enabling consolidation of the target fungal species in the plurality of bioreactor bags, the increasing fungal biomass volume resulting in full spectrum fungal biomass production including target compounds.

20. A method of liquid submerged fermentation using liquid dilution spawning for expanding inoculum of fungal species to produce full spectrum fungal biomass in a production environment, the method comprising:
inoculating growth media in a first sterilized container with a target fungal species of a mother culture for liquid submerged fermentation;
agitating the growth media in the first sterilized container using optimized growth conditions for the target fungal species of the mother culture;
diluting the target fungal species in a second sterilized container by diluting the growth media from the first sterilized container comprising the target fungal species of the mother culture, the diluting the target fungal species using a volume of the growth media from the first sterilized container resulting in a concentration of the target fungal species in the second sterilized container that enables even colonization of the target fungal species in the second sterilized container thereby enabling consolidation of the target fungal species in the second sterilized container; and
inoculating a bioreactor bag for increasing a biomass volume of full spectrum fungal biomass of the target fungal species using a quantity of liquid inoculum from the second sterilized container, the quantity of liquid inoculum from the second sterilized container enabling an even dispersion of fragments of mycelium of the liquid inoculum on a plurality of grain media dispersion points inside of the bioreactor bag, the plurality of grain media dispersion points being a plurality of inoculation points in the bioreactor bag for increasing fungal biomass volume enabling even colonization of the target fungal species in the bioreactor bag thereby enabling consolidation of the target fungal species in the bioreactor bag, the increasing fungal biomass volume resulting in full spectrum fungal biomass production including target compounds.
